(19) United States
(12) Patent Application Publication
(10) Pub. No.: US 2025/0044319 A1
(43) Pub. Date: Feb. 6, 2025

(54) COSMETIC SECUREMENT DEVICE WITH UNIVERSAL COSMETIC HOLDER FOR STABILIZATION OF COSMETIC APPLICATOR CONFIGURED FOR USERS WITH LIMITED MOBILITY

(71) Applicant: L’OREAL, Paris (FR)

(72) Inventors: Casey BARBARINO, San Anselmo, CA (US); Gregoire CHARRAUD, Jersey City, NJ (US); Hyosik Dennis MIN, Palisades Park, NJ (US)

(73) Assignee: L’OREAL, Paris (FR)

(21) Appl. No.: 18/362,520

(22) Filed: Jul. 31, 2023

(51) Int. Cl. G01P 13/00; A45D 44/00

(52) U.S. Cl. CPC G01P 13/00; A45D 44/00; A45D 2200/10; A61B 5/1101

(57) ABSTRACT

A cosmetic securement device for stabilization of a cosmetic applicator, comprising a motion stabilizer handle; and an adapter coupled to a first end of the motion stabilizer handle; wherein the motion stabilizer handle includes at least one sensor configured to detect a movement caused by a user, circuitry configured to determine a compensation movement to offset the detected movement, and at least one motion generation device configured to control a motion of the cosmetic applicator according to the determined compensation movement, and wherein the adapter includes a cosmetic holder configured to hold a plurality of different types of cosmetic applicators, the cosmetic holder comprising a first arm and a second arm, the first arm and the second arm being connected at a hinge and configured to rotate around the hinge.

### FIELD

The present disclosure describes a system and features related to a device for modifying, mitigating, altering, reducing, compensation for, or the like, the movement of a cosmetic applicator caused by unintentional movements, tremors, limited mobility, or the like of a user.

### BACKGROUND

Unintentional movements of the human body, or human tremors, can occur in individuals suffering from motion disorders or even healthy individuals. Due to these unintentional movements, a person may have difficulty in performing a task that requires care and precision, such as applying a cosmetic composition to a part of the body, such as the face, hands, or feet.

Therefore, there is a need for a solution that allows application of a cosmetic composition that is compatible with the diverse and disposable nature of cosmetic applicators.

### SUMMARY

In one embodiment, the present disclosure is directed to a cosmetic securement device for stabilization of a cosmetic applicator, comprising a motion stabilizer handle; and an adapter coupled to a first end of the motion stabilizer handle; wherein the motion stabilizer handle includes at least one sensor configured to detect a movement caused by a user, circuitry configured to determine a compensation movement to offset the detected movement, and at least one motion generation device configured to control a motion of the cosmetic applicator according to the determined compensation movement, and wherein the adapter includes a cosmetic holder configured to hold a plurality of different types of cosmetic applicators, the cosmetic holder comprising a first arm and a second arm, the first arm and the second arm being connected at a hinge and configured to rotate around the hinge.

In one embodiment, the present disclosure is directed to the cosmetic securement device, wherein the first arm includes one or more teeth along an outer wall of the first arm.

In one embodiment, the present disclosure is directed to the cosmetic securement device, wherein the second arm includes one or more divots configured to couple with the one or more teeth of the first arm.

In one embodiment, the present disclosure is directed to the cosmetic securement device, wherein the second arm forms a pawl configured to engage the one or more teeth of the first arm.

In one embodiment, the present disclosure is directed to the cosmetic securement device, wherein the second arm is hollow and the first arm is configured to fit inside the second arm.

In one embodiment, the present disclosure is directed to the cosmetic securement device, wherein the first arm or the second arm is spring-loaded.

In one embodiment, the present disclosure is directed to the cosmetic securement device, wherein an inner wall of the cosmetic holder is lined with a padding material.

In one embodiment, the present disclosure is directed to the cosmetic securement device, further comprising a motor configured to rotate the first arm or the second arm around the hinge.

In one embodiment, the present disclosure is directed to the cosmetic securement device, wherein the first arm is rotated by movement of a magnetic fluid.

In one embodiment, the present disclosure is directed to a cosmetic securement device for stabilization of a cosmetic applicator, comprising a motion stabilizer handle; and an adapter including a cosmetic holder and coupled to a first end of the motion stabilizer handle: wherein the motion stabilizer handle includes at least one sensor configured to detect a movement caused by a user, circuitry configured to determine a compensation movement to offset the detected movement and an aperture of the cosmetic holder based on the cosmetic applicator, and at least one motion generation device configured to control a motion of the cosmetic applicator according to the determined compensation movement, and wherein the cosmetic holder is configured to hold a plurality of different types of cosmetic applicators.

In one embodiment, the present disclosure is directed to the cosmetic securement device, wherein the cosmetic holder includes a first arm and a second arm, the first arm and the second arm being connected at a hinge and configured to rotate around the hinge.

In one embodiment, the present disclosure is directed to the cosmetic securement device, further comprising a motor configured to rotate the first arm or the second arm around the hinge based on the determined aperture.

In one embodiment, the present disclosure is directed to the cosmetic securement device, wherein the first arm includes one or more teeth along an outer wall of the first arm.

In one embodiment, the present disclosure is directed to the cosmetic securement device, wherein the second arm includes one or more divots configured to couple with the one or more teeth of the first arm.

In one embodiment, the present disclosure is directed to the cosmetic securement device, wherein the second arm forms a pawl configured to engage the one or more teeth of the first arm.

In one embodiment, the present disclosure is directed to the cosmetic securement device, wherein the cosmetic holder includes a rotatable ring surrounding one or more internal jaws, wherein the rotatable ring is coupled to a tapered thread along the one or more internal jaws.

In one embodiment, the present disclosure is directed to the cosmetic securement device, further comprising a motor configured to rotate the rotatable ring based on the determined aperture.

In one embodiment, the present disclosure is directed to a cosmetic securement device for stabilization of a cosmetic applicator, comprising a motion stabilizer handle; and an adapter coupled to a first end of the motion stabilizer handle: wherein the motion stabilizer handle includes at least one sensor configured to detect a movement caused by a user, circuitry configured to determine a compensation movement to offset the detected movement, and at least one motion generation device configured to control a motion of the cosmetic applicator according to the determined compensation movement, and wherein the adapter includes a cosmetic holder configured to hold a plurality of different types of cosmetic applicators, the cosmetic holder comprising an adjustable ring.

In one embodiment, the present disclosure is directed to the cosmetic securement device, wherein the cosmetic holder includes a rotatable ring surrounding one or more internal jaws, wherein the rotatable ring is coupled to a tapered thread along the one or more internal jaws.

In one embodiment, the present disclosure is directed to the cosmetic securement device, wherein the adjustable ring is a circular spring.

### BRIEF DESCRIPTION OF THE DRAWINGS

A more complete appreciation of the embodiments and many of the attendant advantages thereof will be readily obtained as the same becomes better understood by reference to the following detailed description when considered in connection with the accompanying drawings, wherein: FIG. 1 shows a conventional motion stabilizing device.

FIG. 2 shows how the conventional motion stabilizing device couples with an adaptor and a make-up applicator.

FIG. 3A shows a diagram of the internal components of a motion stabilizing device according to one embodiment.

FIG. 3B shows a diagram of an alternative embodiment of the motion stabilizing device in which a receiver portion includes an electromagnetic positioner.

FIG. 4 shows an overview of a universal adapter handle connection system.

FIG. 5A shows an illustration of an adapter, according to one embodiment.

FIG. 5B is a transparent side view of the adapter, according to one embodiment.

FIG. 5C is a view of the bottom face of the adapter body, according to one embodiment.

FIG. 5D shows how the adaptor holds a specific cosmetic applicator, according to an embodiment.

FIG. 6A is an illustration of the adaptor 100 with a cutaway view of the adaptor body 110, according to one embodiment.

FIG. 6B is a side view of the adaptor with a cutaway view of the adaptor body, according to one embodiment.

FIG. 6C is a transparent view of the outer shell of the adaptor, according to one embodiment.

FIG. 6D is a transparent view of the inner body of the adaptor, according to one embodiment.

FIG. 7 shows a block diagram of the communicative hardware elements in the stabilizer device and the adapter, according to an embodiment.

FIG. 8 shows different protocols for when the adapter has different cosmetic applicators, according to an embodiment.

FIG. 9 shows a flowchart for performing the auto-detection.

FIG. 10 shows an adjustable cosmetic adapter.

FIG. 11 shows an adjustable cosmetic adapter including ratcheting teeth.

FIG. 12 shows an adjustable cosmetic adapter including a spring-loaded ring.

FIG. 13 shows an adjustable cosmetic adapter with a manual aperture adjustment.

### DETAILED DESCRIPTION

The present disclosure describes a cosmetic applicator system that minimizes, modifies, mitigates, alters, reduces, compensates for, or the like unintentional movements by stabilizing, orienting, operating, controlling, etc. an applicator for a user and is also designed to be flexible to accommodate different types of commercially available cosmetic applications. The present disclosure further describes a system and features to enhance the functionality of such a cosmetic applicator system.

The basic features and operation of a motion stabilizing device for a cosmetic applicator is described in U.S. Pat. No. 11,458,062, which is incorporated herein by reference.

FIG. 1 shows a conventional motion stabilizing device 1100, which serves as a base unit for receiving a cosmetic applicator according to an embodiment. The device 1100 includes a handle portion 1101, a receiver portion 1102 and a strap 1103. The receiver portion 1102 includes an interface 1104, shown as a male connector that couples with a cosmetic applicator, which will be discussed in detail below. The receiver portion could be utilized for communication between the base unit and the applicator. The connection to an adaptor and/or an applicator could be accomplished with a mechanical coupling, such as screw-in or snap-fit, or it could be accomplished with magnets.

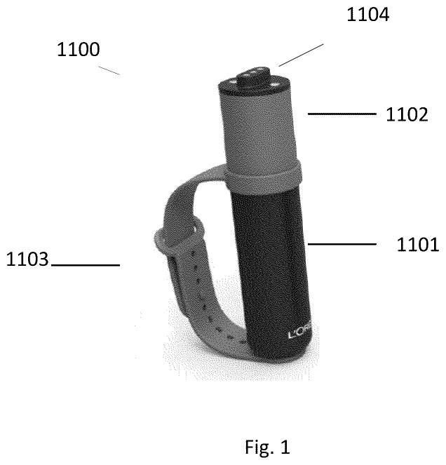

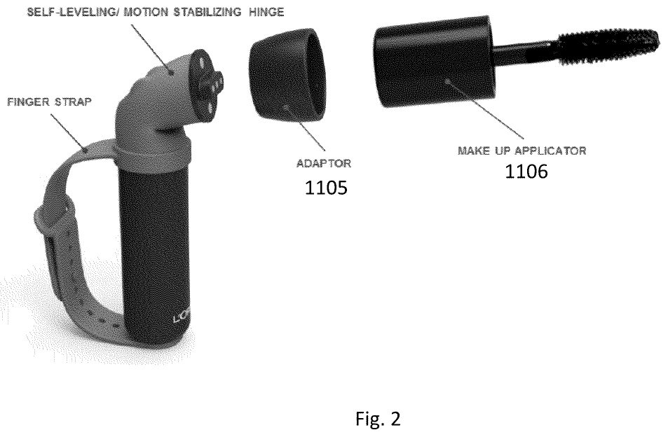

FIG. 2 shows how the device 1100 couples with an adaptor 1105 and a make-up applicator 1106. It can be seen that the adaptor fits over the exposed end of the receiver portion 1102. The adaptor includes electrical mating connectors (a female connector-not shown) in a recessed portion to make contact with the electric interface of the receiver portion 1101.

As shown in FIG. 2, the receiver portion 1102 is configured to contort, articulate, reposition, etc., between an upright posture (as shown in FIG. 1) and an angled posture (as shown in FIG. 2). This is accomplished with a hinge mechanism contained inside the receiver portion 1102. FIG. 2 shows that the hinge mechanism is a self-leveling/motion stabilizing hinge.

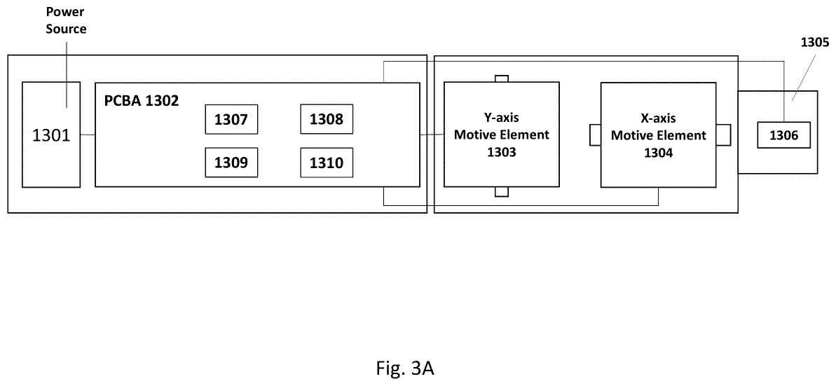

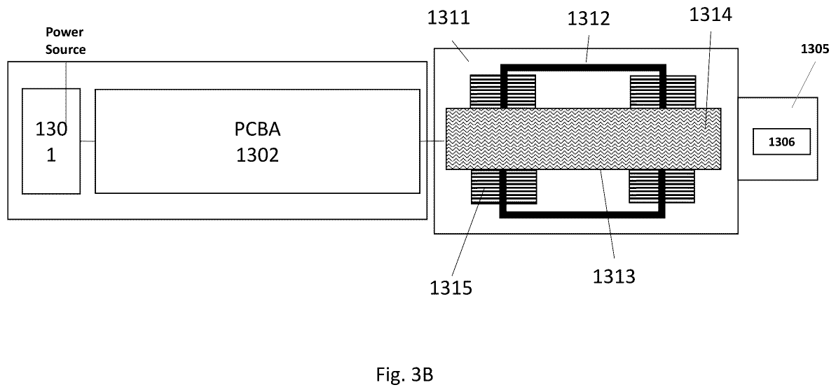

FIG. 3A shows a diagram of the internal components of device 1100 according to one embodiment. In the handle portion, the device includes a power source 1301, which may be a battery or the like. The device includes a printed circuit board assembly (PCBA) 1302, which may include positional sensor circuitry 1307, reader circuitry 1308, control circuitry 1309, and communication interface 1310, as understood in the art.

For instance, as the sensor circuitry 1307, the PCBA may include at least one inertial sensor and at least one distributed motion sensor to detect unintentional muscle movements and measure signals related to these unintentional muscle movements that are created when a user adversely affects motion of the applicator. These sensors also detect the motion of the stabilized output relative to device. The control circuitry sends voltage commands in response to the signals to the motion generating elements (described below) to cancel the user's tremors or unintentional muscle movements. This cancellation maintains and stabilizes a position of the applicator, keeping it stable.

One of ordinary skill in the art readily recognizes that a system and method in accordance with the present invention may utilize various implementations of the control circuitry and the sensor circuitry and that would be within the spirit and scope of the present invention. In one embodiment, the control circuitry 1309 comprises an electrical system capable of producing an electrical response from sensor inputs such as a programmable microcontroller or a field-programmable gate array (FPGA). In one embodiment, the control circuitry comprises an 8-bit ATMEGA8A programmable microcontroller manufactured by Atmel due to its overall low-cost, low-power consumption and ability to be utilized in high-volume applications.

In one embodiment, the at least one inertial sensor in the sensor circuitry is a sensor including but not limited to an accelerometer, gyroscope, or combination of the two. In one embodiment, the at least one distributed motion sensor in the sensor circuitry is a contactless position sensor including but not limited to a hall-effect magnetic sensor.

The system created by the combination of the sensor circuitry, the control circuitry, and the motion generating elements may be a closed-loop control system that senses motion and acceleration at various points in the system and feeds detailed information into a control algorithm that moves the motion-generating elements appropriately to cancel the net effect of a user's unintentional muscle movements and thus stabilize the position of the applicator. The operation and details of the elements of the control system and control algorithm are understood in the art, as described in U.S. PG Publication 2014/0052275A1, incorporated herein by reference.

The communication interface 1310 may include a network controller such as BCM43342 Wi-Fi, Frequency Modulation, and Bluetooth combo chip from Broadcom, for interfacing with a network.

In the receiver portion of the device, there may be two motive elements to allow 3-dimensional movement of the receiver as anti-shaking movement. The two motive elements include a y-axis motive element 1303 and an x-axis motive element 1304, each being connected to and controlled by the PCBA 1302. Each of the motive elements may be servo motors as understood in the art. The device further includes end effector coupling 1305, which is configured to couple with the adaptor 1105. The end effector coupling 1305 may include a radiofrequency identification (RFID) reader 1306, configured to read an RFID tag, which may be included with the applicator, as will be discussed below.

FIG. 3B shows a diagram of an alternative embodiment of the device 1100 in which the receiver portion includes an electromagnetic positioner 1311 instead of the motive elements shown in FIG. 3A. The electromagnetic positioner 1311 may include U-shaped magnetic cores 1312 arrayed around a non-magnetic tube 1313, which is filled with a magnetic fluid 1314.

Each of the magnetic cores has arm portions that are surrounded by windings 1315. The magnetic cores may be controlled by the control circuitry in the PCBA 1302 to act as a controllable active magnetic field-generating structure which is used to generate a variable magnetic field that acts upon the magnetic fluid, causing it to be displaced, thereby enabling the armature to be moved to a desired coordinate position and/or orientation. The details of implementing the electromagnetic positioner 1311 may be found in U.S. Pat. No. 6,553,161, which is incorporated herein by reference.

In the above-described conventional motion stabilizing device, there is a problem that the interface 1104 that receives the adaptor 1105 requires a specific point of attachment to align properly with the interface.

Therefore, the below embodiments provide a universal adapter connection between the handle of the motion stabilizing device in order to improve user experience and reduce the struggle and time taken to set up the system for use.

In one embodiment, the present disclosure is directed towards a cosmetic applicator. The cosmetic applicator can be used for a variety of cosmetics and cosmetic applications, including, but not limited to, mascara, eyeliner, eyebrow products, lip products (lipstick, lip gloss, lip liner, etc.), skin products, and/or hair products. In one embodiment, the cosmetic applicator can include an adapter, wherein the adapter can connect the cosmetic applicator to a motion stabilizer. The motion stabilizer can be, for example, a handle that can counteract unintentional motions such as tremors or spasms. These motions can interfere with the application of cosmetics and can also make it difficult to generally interact with cosmetic applicators or tools. For example, the many cosmetic products require a twisting motion or force to be applied to open or extrude the product. It can be difficult for users to achieve the range of motion or the precision necessary to apply these forces to the cosmetic. In one embodiment, the cosmetic applicator can hold a cosmetic and can enable the proper force to be applied to the cosmetic to open, close, mix, stir, blend, extrude, or achieve other similar functions necessary for application.

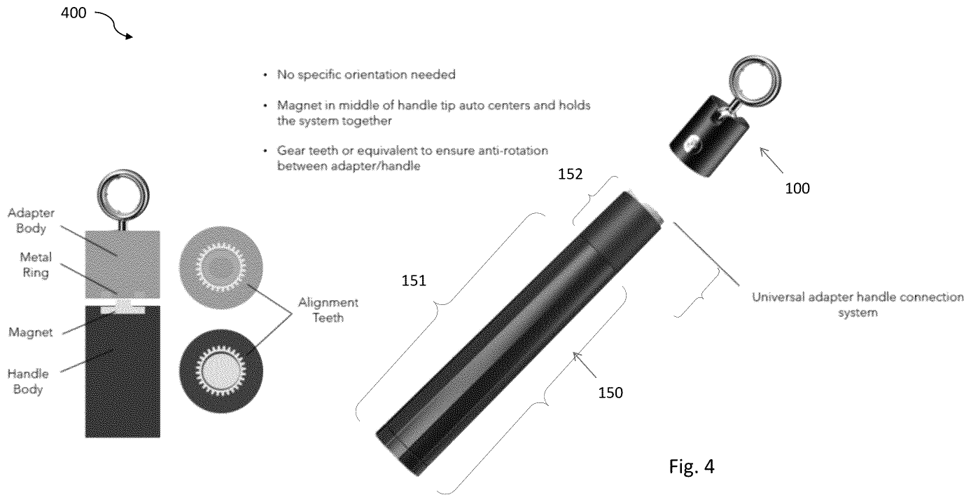

FIG. 4 shows an overview of a cosmetic securement device including a motion stabilizer device 150 and a cosmetic adapter 100, according to one embodiment of the present disclosure. The motion stabilizer device 150 can include at least one of a handle portion 151 and a hinge portion 152 (receiver portion). In an embodiment, the handle portion 151 is functionally similar to the handle portion 1101 shown in FIG. 1. The receiver portion 152 can be functionally similar to the receiver portion 1102 shown in FIG. 1. In an embodiment, the motion stabilizer device 150 as a whole is referred to herein as a motion stabilizer handle. In an embodiment, the cosmetic securement device includes a cosmetic adapter 100, wherein the adapter 100 can be coupled to the receiver portion 152 of the motion stabilizer device 150. In an embodiment, the cosmetic adapter 100 holds a cosmetic tool or product. In one embodiment, the cosmetic adapter 100 can be a universal adapter configured to hold different types of cosmetic products. For example, the adapter 100 can include a ring-shaped holder. Various configurations of the adapter 100 can be used for different cosmetics and tools. For example, the shape and dimensions of the cosmetic holder can be configured for different cosmetic products. The adapter 100 can be easily attached to and removed from the motion stabilizer device. The basic features and operation of the cosmetic securement device of FIG. 4 are described in co-pending U.S. patent application Ser. No. 18/091,882; 18/091,920; 18/091,843; 18/091,925; 18/148,957; and Ser. No. 18/148,880; and Ser. No. 18/148,930, which are incorporated herein by reference.

In one embodiment, the adapter 100 is coupled to the receiver portion 152 of the motion stabilizer device 150 via a magnetic attachment. The base or bottom end of the adapter 100 can form a chamber that can fit over a projection at the tip of the receiver portion 152. The base of the adapter can include at least one magnet, wherein the at least one magnet can be attracted to and attach to a magnet in the receiver portion 152. In one embodiment, the chamber formed at the base of the adapter 100 can fit over the receiver portion 152 in more than one orientation. The chamber can be a hollow area within the body of the adapter that is fully contained by the walls of the adapter 110. The chamber can be approximately conical in shape. In one embodiment, the chamber is cylindrical. The chamber can be configured to fit over a projection on one end of the motion stabilizer. The chamber can be used to align and guide the attachment of the cosmetic applicator to the motion stabilizer. The fit of the chamber over and around the projection on the end of the motion stabilizer limits lateral motion that would misalign the cosmetic applicator and the motion stabilizer. It can be easier for a user to align the chamber over the end of the motion stabilizer than it would be for the user to align the edges of a circular face of the adapter with the edges of a circular face of the motion stabilizer. The chamber can have rotational symmetry such that the cutout can be placed over the end of the motion stabilizer in any orientation or at any degree of rotation around the axis of the chamber. In one embodiment, the bottom end of the adapter body can include additional physical structures that can align, guide, and fix the adapter to the motion stabilizer.

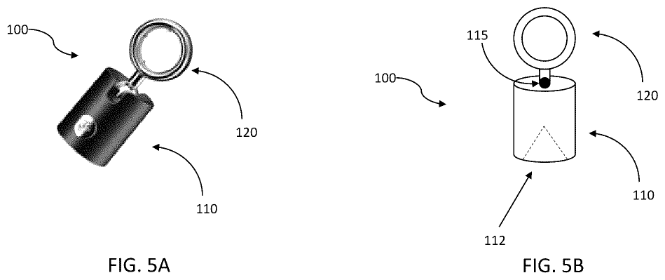

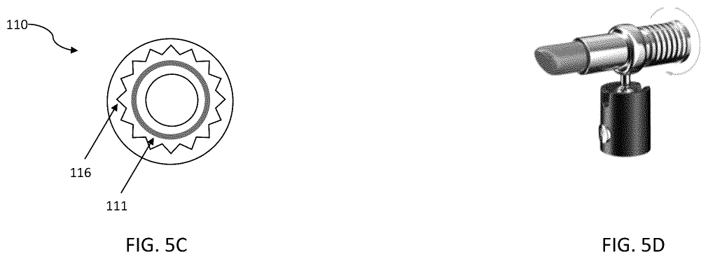

FIG. 5A shows an illustration of adapter 100, according to one embodiment of the present disclosure. The adapter 100 can include an adapter body 110, wherein the adapter body 110 can be connected to the motion stabilizer 150. The adapter body 110 can be connected to or can form the base or body of a cosmetic holder 120. Each of these components will be discussed in further detail herein. Various configurations of the adapter 100 can be used for different cosmetics and tools. For example, the shape and dimensions of the cosmetic holder 120 can be configured for different cosmetic products. The adapter 100 can be easily attached to and removed from the motion stabilizer, as will be discussed herein. In one embodiment, the adapter 100 can broaden the range of motion of a cosmetic applicator to enable movements that are necessary for cosmetic application. In some embodiments, the adapter 100 can be attached to a stand or carriage for further actions to be performed with a cosmetic held by the cosmetic applicator.

FIG. 5B is a transparent side view of the adapter 100, according to one embodiment of the present disclosure. The adapter body 110 can be shaped approximately as a right cylinder. A first end of the adapter can be attached to one end of a motion stabilizer. The motion stabilizer can be a cylindrical rod forming a handle for a user to grasp. The first end can also be referred to hereinafter as a bottom end or a base of the adapter. The bottom end of the adapter body 110 can include at least one magnet, wherein the at least one magnet can be attracted to and attach to a magnet in the motion stabilizer. The bottom end of the adapter body 110 can include additional physical structures that can align, guide, and fix the adapter to the motion stabilizer. In one embodiment, the bottom end of the adapter body 110 can form a chamber 112, as illustrated in the transparent view of FIG. 5B. The chamber can be a hollow area within the body of the adapter 110 that is fully contained by the walls of the adapter 110. The chamber 112 can be approximately conical in shape. In one embodiment, the cutout can be cylindrical. The chamber 112 can be configured to fit over a projection on one end of the motion stabilizer. The chamber 112 can be used to align and guide the attachment of the cosmetic applicator to the motion stabilizer. The fit of the chamber 112 over and around the projection on the end of the motion stabilizer limits lateral motion that would misalign the cosmetic applicator and the motion stabilizer. It can be easier for a user to align the chamber 112 over the end of the motion stabilizer than it would be for the user to align the edges of the circular face of the adapter with the edges of the circular face of the motion stabilizer. The chamber 112 can have rotational symmetry such that the cutout can be placed over the end of the motion stabilizer in any orientation or at any degree of rotation around the axis of the chamber 112. In one end, a magnet can be fixed at the tip of the chamber, inside the body of the adapter. The magnet at the tip of the cutout can be attached to a magnet at the tip of the projection on the end of the motion stabilizer.

The cosmetic holder 120 can be located at a second end of the cosmetic applicator. In one embodiment, the cosmetic holder 120 can be a ring, wherein a cosmetic tool or product can be inserted into the ring. Many cosmetic tools, such as mascara wands, lipstick applicators, concealer applicators, and cosmetic pencils, are approximately cylindrical. In one embodiment, the ring can secure the cosmetic tools at the widest part of the tool. A ring-shaped holder can also hold cosmetic tools of varying shapes, such as cuboids. In one embodiment, the cosmetic holder 120 can include one or more projections along the inner wall of the holder and extending outward into the center of the holder. Contact and friction between the one or more projections and an inserted cosmetic tool can result in a more secure grip or hold of the cosmetic holder around the inserted cosmetic tool. In some embodiments, the inner wall of the cosmetic holder 120 can be lined with a material that can improve the grip or hold of the cosmetic holder around the inserted cosmetic tool. The improved grip can be a result of a material property, such as a coefficient of friction or a material deformation. For example, the inner wall can be lined with rubber or silicon. In one embodiment, the lining material can be a textured material with grooves, ridges, bumps, or similar features that prevent movement or slippage of the cosmetic tool once it is inserted into the cosmetic holder 120.

Various cosmetic holder configurations are compatible with the cosmetic applicator 100. In one implementation, the holder can be an open ring or a C-shaped holder. In one embodiment, the holder can be a loop, wherein the ends of the loop can overlap. In one embodiment, the holder can be one or more coiled loops. In one embodiment, the cosmetic holder 120 can be a tube or sheath forming a chamber inside the tube, wherein a cosmetic tool or product can be inserted into the chamber. In one embodiment, the tube can be closed on one end to contain a cosmetic tool or product in the chamber. In one example, the tube can be approximately cylindrical. In one example, the sheath can be a partial cylinder, such as a half-cylinder that does not wrap fully around the inserted cosmetic. In one embodiment, the size of the cosmetic holder 120 can be modified. For example, the cosmetic holder 120 can be a round holder, wherein the diameter of the holder can be modified to accommodate different cosmetic tools. A pushing force on a point along the outside of the holder can compress the holder to that of a smaller diameter. A pulling force on a point along the inside of the holder can expand the holder to that of a larger diameter. In one embodiment, the force can be applied at a single point along the holder to enable one-handed modification of the holder diameter. In one implementation, a user can be provided with a number of cosmetic holders, wherein each cosmetic holder has different dimensions or shapes for different cosmetic tools or products. A cosmetic holder 120 can be removed from the adapter body 110 and replaced with another of suitable configuration.

In one embodiment, the cosmetic holder 120 can be attached to the adapter body 110 with a joint 115, such as a ball joint. The ball joint can enable movement of the cosmetic holder 120 relative to the adapter body 110. For example, the cosmetic holder 120 can be rotated in place or can pivot to form an angle with the adapter body 110. The joint can be configured for continuous motion of the cosmetic holder 120 or for discrete steps of movement. According to some embodiments, the motion of the cosmetic holder 120 can be constrained. For example, the adapter 110 can form a walled channel, wherein the joint is disposed inside the walled channel. The walls of the channel can constrain movements of the joint and/or the cosmetic holder 120. In one embodiment, the joint 115 can be a motorized component. The cosmetic holder 120 can move as a result of the motorized component without requiring external force from a user.

FIG. 5C is a view of the bottom face of the adapter body 110, according to one embodiment of the present disclosure. The bottom face of the adapter body 110 can be approximately a circle given that the adapter is a right cylinder. A magnet 111 can be disposed on the bottom face of the adapter. In one embodiment, the magnet 111 can be a uniformly ring-shaped magnet, as illustrated in FIG. 5C. For example, the magnet 111 can have the shape of a washer. The ring-shaped magnet 111 can be attracted to and attach to a magnet disposed along the circumference of one end of the motion stabilizer. In some embodiments, the adapter body 110 can be attached to the motion stabilizer in any orientation because of the ring-shaped magnet 111. The adapter body 110 can be attached to the motion stabilizer with any rotation of the adapter 100 around the central axis of the adapter. In one embodiment, the adapter 100 can be a universal adapter and can attach a magnetic end of the motion stabilizer in any orientation regardless of the positioning and/or polarity of magnets at the end of the motion stabilizer. Any portion of the ring-shaped magnet 111 at the base of the adapter can contact the magnets on the motion stabilizer to attach the adapter to the motion stabilizer.

The ring-shaped magnet 111 of the present disclosure presents an advantage over traditional adapters for motion stabilizer. For example, certain motion stabilizers can include two magnets located diametrically opposite to each other on a circular face of the motion stabilizer. Most traditional adapters include two magnets at the base for attachment to a motion stabilizer. The adapter can only be attached to the motion stabilizer if the two magnets on the motion stabilizer align with the two magnets in the same position on the adapter. In some implementations, the polarity of the magnets on the motion stabilizer can also differ such that each magnet on the motion stabilizer will only be attracted to one of the two magnets on the adapter. The limitations on the orientation of the traditional adapter can pose issues for users attempting to attach the adapter to the motion stabilizer. For example, users may lack the precision of movement needed to align the magnets as needed for attachment. In addition, the limitations on the orientation of the traditional adapter means that the orientation of the tool or instrument that is held by the applicator is also limited. When applying cosmetic products to or near the face, it can be desired that the applicator can be fixed at various angles and orientations or that the orientation of the applicator can be adjusted. For example, certain mascara wands are curved. The mascara wand should contact the eyelashes such that the curvature of the wand conforms to the curvature of the eye. It can be very difficult to achieve this position if the adapter holding the mascara wand can only be attached to the motion stabilizer in one orientation, as with traditional adapters with a limited number of magnets in set positions. In contrast, an adapter with a ring-shaped magnet, as in FIG. 5C, can be attached to the motion stabilizer in any orientation. The adapter can be rotated until the mascara wand, or any other cosmetic tool, is in a desired orientation for application and then attached to the motion stabilizer with contact between the ring-shaped magnet and any magnets disposed on the face of the motion stabilizer. The motion stabilizer can be maneuvered in various positions while the attached applicator and cosmetic also move. For example, the orientation of the attached applicator and the cosmetic can change as the user applies the cosmetic. The attachment of the applicator to the motion stabilizer without orientation expands the range of movement of the attached cosmetic.

In one embodiment, the adapter body 110 can include a ring of gears, teeth, or similar alignment structures 116. The alignment structures 116 can be located at the base of the adapter and can be concentric with the ring-shaped magnet 111. In one embodiment, the alignment structures can be located along the inner wall of the cutout at the bottom end of the adapter. In some embodiments, the alignment structures 116 can fit into or interlock with corresponding alignment structures on the motion stabilizer when the adapter is attached to the motion stabilizer. The alignment structures 116 can limit the rotation of the cosmetic applicator once the adapter has been attached to the motion stabilizer. Certain motion stabilizers can rotate in order to counteract any rotational force applied to the motion stabilizer so that an attachment to the motion stabilizer (such as the cosmetic applicator) remains steady. The “anti-rotation” mechanism can be useful for users who experience sudden, involuntary movements while holding the motion stabilizer. In one embodiment, the alignment structures 116 can be a ring of raised nubs or teeth-like structures that can fit into corresponding grooves or receptacles at the end of the motion stabilizer that is attached to the base of the adapter. In one embodiment, the alignment structures 116 can be a ring of recessed structures or a combination of raised and recessed structures that can fit into corresponding raised and/or recessed structures at the end of the motion stabilizer. The fitting of the alignment structures 116 into complementary structures at the end of the motion stabilizer can fix the cosmetic applicator in place when the adapter has been attached to the motion stabilizer. Any rotation of the motion stabilizer in order to counteract involuntary user movement will thus be carried through to the cosmetic applicator to maintain the orientation of the cosmetic applicator once the cosmetic applicator is attached to the motion stabilizer.

The alignment structures 116 can provide a range of discrete orientations at which the adapter can be attached to the motion stabilizer. For example, the adapter can only be attached to the motion stabilizer when each of the alignment structures 116 of FIG. 5C is aligned with a corresponding recessed structure in the motion stabilizer. If the alignment structures 116 are not aligned with and sitting in the recessed structures, the adapter will not be properly attached to the motion stabilizer. However, the symmetry of the repeated alignment structures 116 can still enable the attachment of the adapter at a number of rotations (e.g., 10+ rotational options) for easy attachment.

FIG. 5D shows how the adaptor 100 holds a specific cosmetic applicator, such as a lipstick applicator within the cosmetic holder 120. While a lipstick applicator is shown, the cosmetic holder can hold numerous examples of applicators, such as mascara applicators and nail polish applicators as other examples. In contrast to the adapter 1105 of FIG. 1, it can be seen that the cosmetic holder 120 holds the applicator at perpendicular angle to the handle of the motion stabilizer 150. This provides a more relaxed and ergonomic hand position for the user when using the cosmetic applicator.

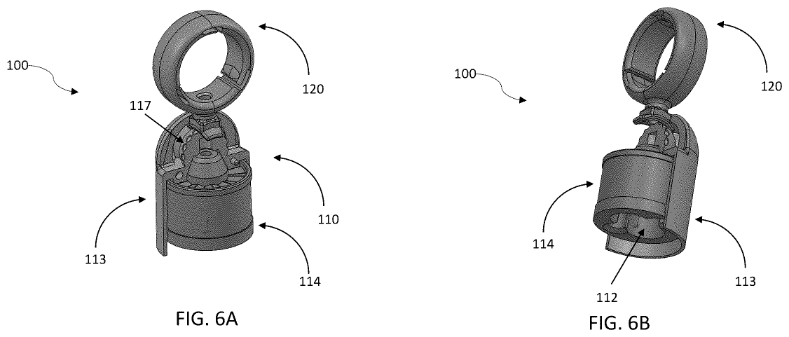

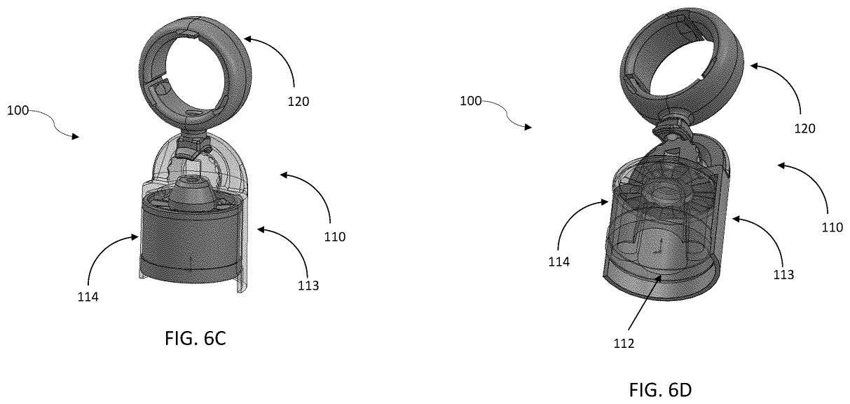

FIG. 6A is an illustration of the adaptor 100 with a cutaway view of the adaptor body 110, according to one embodiment of the present disclosure. In one embodiment, the adaptor body 110 can include an outer shell 113, wherein the outer shell 113 encloses an inner body 114. The inner body 114 can be attached to a motion stabilizer via the magnetic attachment described herein with reference to FIG. 5C. In one embodiment, the cosmetic holder 120 can be attached to the outer shell 113. The attachment of the cosmetic holder 120 to the outer shell 113 can enable movement of the cosmetic holder 120 that is independent of the movement of the inner body or the motion stabilizer. The attachment of the cosmetic holder 120 to the outer shell 113 can be enclosed within the adaptor body 110. In one embodiment, the cosmetic holder 120 can be attached to the outer shell 113 along a series of detents in the outer shell. The detents can be, for example, a series of indentations, grooves, teeth, or the like, such as the indented structures 117 of FIG. 6A. The position of the cosmetic holder 120 can change by moving the cosmetic holder 120 linearly along the series of detents, such that the cosmetic holder 120 can be fixed in place at each of the detent structures 117. In one embodiment, the base of the cosmetic holder 120 can include a projection that fits into each of the detent structures 117. The projection can snap into place when positioned at or against each of the detent structures 117 to temporarily lock the cosmetic holder 120 into an angled position within a single plane. The plane of the face (opening) of the cosmetic holder 120 can stay the same while the angular position of the cosmetic holder 120 can be adjusted. A force can be applied by a user to position the cosmetic holder 120 at any of the detent structures 117. The cosmetic holder 120 can thus be angled relative to the central axis of the adaptor body 110 and the attached motion stabilizer. In one embodiment, the cosmetic holder 120 can be attached to the adaptor body 110 with a 2-axis adapter. The angle of the cosmetic holder 120 can be adjusted as described with reference to the detent structures 117. Additionally or alternatively, the plane of the opening of the cosmetic holder 120 can also be adjusted by a rotation of the cosmetic holder 120 for 2-axis movement. For example, the cosmetic holder 120 can rotate axially at or near the point of attachment to the adaptor body 110. In one embodiment, the rotation of the cosmetic holder 120 can be modulated with detents or similar structures to provide a number of fixed rotational positions of the cosmetic holder 120. For example, the cosmetic holder 120 or the adaptor body 110 can include an internal gear or ratcheting structure, wherein the cosmetic holder 120 can be rotated a number of degrees to engage the teeth of the internal gear. The cosmetic holder 120 can be temporarily locked in a rotational position when the teeth are engaged.

FIG. 6B is a side view of the adaptor 100 with a cutaway view of the adaptor body 110, according to one embodiment of the present disclosure. The inner body 114 of the adaptor body 110 can form the chamber 112, as has been described herein. Various shapes and dimensions of the chamber 112 are compatible with the present adaptor 100. In one embodiment, the outer shell 113 can extend past the end of the inner body 114. The outer shell 113 can surround the attachment point of the adaptor 100 to a motion stabilizer or other compatible structure and can prevent the adaptor 100 from being displaced from the attached structure. In some embodiments, the magnetic ring can be formed at the base of the inner body 114.

FIG. 6C is a transparent view of the outer shell 113 of the adaptor 100, according to one embodiment of the present disclosure. The outer shell 113 can form a cavity surrounding the inner body 114. In one embodiment, the inner body 114 can rotate relative to the outer shell 113 while the outer shell 113 and/or the cosmetic holder 120 are fixed in place and do not rotate. For example, the inner body 114 can rotate in order to counteract an unintentional rotational movement of the motion stabilizer or the adaptor 100. The rotation of the inner body 114 is not translated to the cosmetic holder 120 because the cosmetic holder 120 is not in contact with the inner body 114. Similarly, the fitting of the outer shell 113 around the inner body 114 can include a gap between the outer shell 113 and the walls of the inner body 114 so that the rotation of the inner body 114 is not translated to the outer shell 113.

FIG. 6D is a transparent view of the inner body 114 of the adaptor 100, according to one embodiment of the present disclosure. The inner shell 114 can form the chamber 112, as has been described herein. The chamber 112 can extend to the top of the inner body. In one embodiment, the chamber 112 can be shaped as a central cylinder with two cylinders adjacent to the central cylinder, as illustrated in FIG. 6D. The shape of the chamber 112 and the symmetry of the chamber 112 can depend on the intended attachment to a motion stabilizer or other structure. In one embodiment, the shape of the chamber 112 can depend on the desired movement of the adaptor 100. For example, an infinitely symmetrical chamber can be attached onto a motion stabilizer in any orientation and can remain stationary when the motion stabilizer rotates. In another example, a chamber with limited axes of symmetry can result in a fixed number of orientations at which the adaptor can be attached to the motion stabilizer.

In one embodiment, the attachment of the cosmetic applicator to the motion stabilizer can activate at least one sensor to indicate that the adapter body 110 has been successfully attached to the motion stabilizer. The at least one sensor can be, for example, a Hall effect sensor that can detect and respond to the magnetic field produced by the adapter and/or the motion stabilizer. In one embodiment, the at least one sensor can be a pressure-activated sensor. Alternative sensors known to those of ordinary skill in the art can be compatible with the systems and apparatuses disclosed herein. In one embodiment, the at least one sensor can detect when the cosmetic applicator has not been attached to the motion stabilizer properly. For example, a Hall effect sensor can detect a partial magnetic field from the adapter with a magnitude that is less than an expected magnitude. The partial magnetic field can indicate that a portion of the ring-shaped magnet on the adapter is physically shielded or has failed to produce a magnetic field. In one embodiment, the data from the at least one sensor can be used to notify a user to inspect the cosmetic applicator or reattach the cosmetic applicator.

In one embodiment, the adapter 100 can include one or more sensors and/or an integrated circuit. The one or more sensors can include, but are not limited to, pressure sensors, light sensors, weight sensors, tension or force sensors, a Hall sensor, an accelerometer, a gyroscope, or the like. Data from the one or more sensors can be used to identify when a cosmetic has been inserted into or attached to the cosmetic applicator. In one embodiment, the data from the one or more sensors can be used to identify physical features of an attached cosmetic, such as a size, a shape, or a weight of the attached cosmetic. In one embodiment, the data from the one or more sensors can relate to a motion of the cosmetic applicator 100 or a motion of an attached component, such as the attached cosmetic or the motion stabilizer. In one embodiment, the data from the one or more sensors can be transmitted from the cosmetic applicator to a remote device, such as a server or a user device. In one embodiment, the cosmetic applicator can receive data, including instructions, from the same remote device or a second remote device. Further detail surrounding network

In an embodiment, the system described above includes the functionality to auto-detect adapters to activate specific protocols to the motion stabilizer device 150. For instance, the motion stabilizer device 150 may perform auto detection of different adapters that are used for different cosmetics such as lipstick, mascara, etc. As soon as the adapter is connected to the handle of the motion stabilizer device 150, the handle will automatically recognize which style of adapter (for example, lipstick vs mascara) is attached and will load a specific set of protocols developed for that specific application.

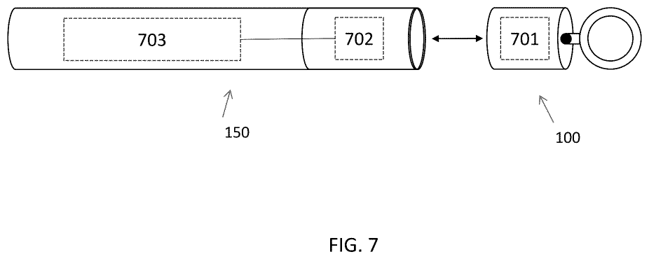

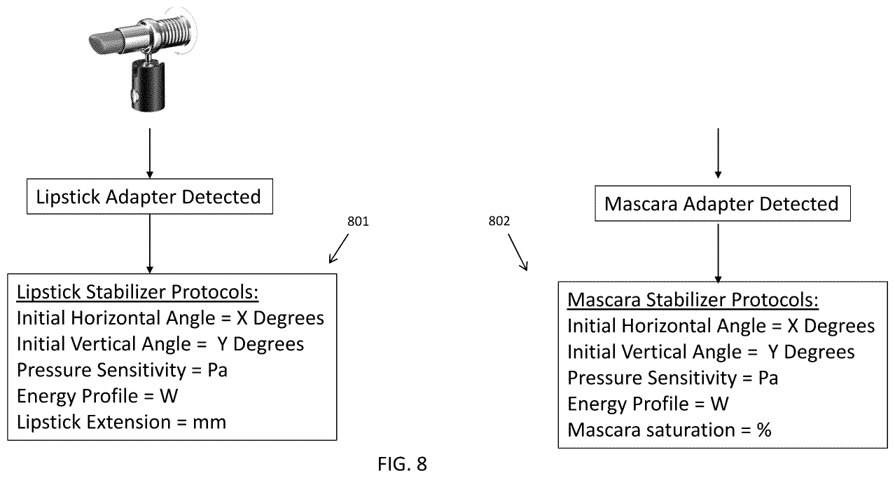

FIG. 7 shows a block diagram of the communicative hardware elements in the stabilizer device 150 and the adapter. The adapter includes a communication interface 701 and the receiver portion of the stabilizer device includes a communication interface 702. The communication interfaces, in one example, is a near field communication (NFC) interface (where element 702 is a radiofrequency identification (RFID) reader configured to read an RFID tag 701). Furthermore, the receiver portion of the stabilizer device includes the hall effect sensor 704 described above. Thus, the RFID reader may be activated in response to the hall effect sensor 704 detecting insertion of the adapter 100. Alternatively, each of interfaces 701 and 702 operate as Bluetooth interfaces in another example, such as Bluetooth Low Energy (BLE) type interfaces.

The interface 702 in the receiver portion is connected to PCBA 703 that is coupled to or includes a memory 705 that stores different protocol information.

Once device 150 recognizes the adapter 100 that is present, it will receive information on the type of specific adapter it is based on information stored on the communication interface 701. The communication interface 702 then communicates the information to the PCBA 703, which stores specific protocols in the device software that target specific gestures and movements for the specific adapter connected (i.e. if lipstick adapter is attached, device will call on lipstick gesture protocols)

For instance, as shown in FIG. 8, a first set of protocols 801 for a lipstick may include specific parameters initial angles of orientation of the stabilizer along with settings for pressure sensitivity, energy consumption, and initial lipstick extension. While a different second set of protocols 802 for a mascara may include different specific parameters initial angles of orientation of the stabilizer along with settings for pressure sensitivity, energy consumption, and mascara saturation.

There could be multiple adjustments to each protocol depending on the adapter and applicator. For example, with lipstick it is desired to remove the axial rotation of the adapter with respect to the handle since this can at times cause unwanted or unexpected movements for the user. It is also desired to limit the amount of flexion, and to create an initial “home position” as the hand holding position may be different for lipstick vs mascara. Additionally, it is desired to change the speed at which the motors move for mascara to be a bit slower so that there is less risk of unintended movements within close vicinity of the eyes. An example for additional lipstick protocol features are as follows.
Disable axial rotation of adapter relative to handle.
Set motor speed to 100% for quickest possible adjustments.
Flex adapter to initial “home” angular setting for best application orientation

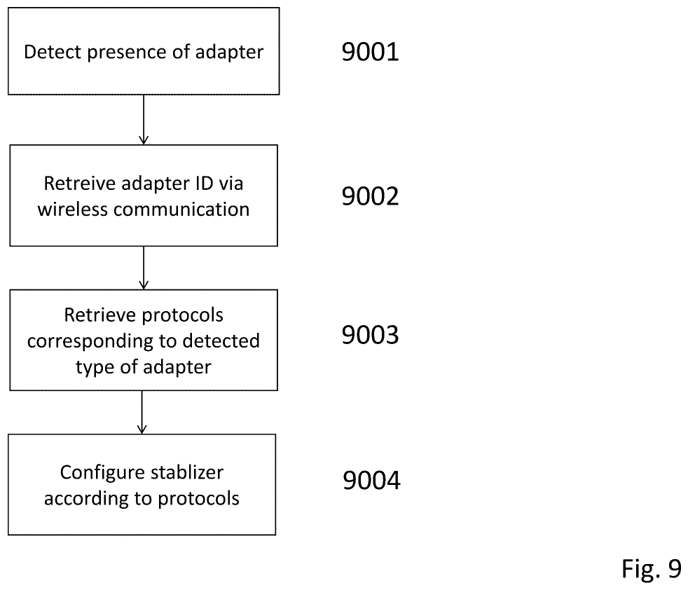

FIG. 9 shows a flowchart for performing the auto-detection as discussed above. The steps are performed at the motion stabilizer device 150. In step 9001, the communication interface 702 detects the presence of the adapter 100. For instance, when the communication interface 702 is an RFID reader and the communication interface 701 is an RFID tag, these elements may perform an initial handshake before establishing a communication channel, as is understood in the art.

In step 9002, after a communication channel is established between the communication interfaces 701 and 702, the communication interface 701 transmits identification information to the communication interface 702. The identification information indicates the type of adapter as discussed above.

In step 9003, the communication interface 702 transmits the received identification information to the PCBA 703 that is onboard the stabilizer device, and the PCBA 703 retrieves the protocols stores on a local memory that correspond to the identified type of adapter.

In step 9003, the PCBA 703 controls configuration of the motion stabilizer device according to the stored protocols after it is detected that the adapter is attached to the motion stabilizer device.

With the above procedures, not only will the motion stabilizer device recognize an adapter is present, but it will recognize whether it is a specific adapter used for lipstick, mascara, or others. Once device recognizes the adapter that is present, it will call on specific protocols in the device software that target specific gestures and movements for the specific adapter connected (i.e. if lipstick adapter is attached, device will call on lipstick gesture protocols).

In one embodiment, the size of the opening (aperture) of the cosmetic holder 120 can be adjustable in order to provide a more universal cosmetic applicator holder that can secure a wide variety of cosmetic tools and products, which can vary greatly in size. Companies can manufacture the same type of cosmetic product with different dimensions for different product lines or stock keeping units (SKUs). It can be preferable for the cosmetic holder 120 to be adjustable while maintaining a secure fit and range of motion that is provided by an attached motion stabilization device 150 and any rotational mechanisms. In one embodiment, the cosmetic holder 120 can expand or contract to fit around an inserted cosmetic tool. In one embodiment, the aperture of the cosmetic holder 120 can be self-adjusting to minimize user interaction, which can be difficult for users with limited mobility or fine motor skills. In one embodiment, the aperture can be adjusted using a button, switch, tab, strap, gear, or similar mechanical component. It can be easier for a user to actuate a mechanical component that is connected to the cosmetic holder 120 rather than directly adjusting the cosmetic holder 120. The aperture of the cosmetic holder 120 can be adjustable for a ring-shaped cosmetic holder 120, as illustrated in FIG. 5. The mechanisms described herein can also be compatible with a C-shaped cosmetic holder, an elliptical or oval-shaped cosmetic holder, a cylindrical cosmetic holder, etc.

In one embodiment, the cosmetic holder 120 can include an elastic or spring-loaded band, wherein the band can stretch to widen the aperture of the cosmetic holder 120 and can retract to narrow the aperture of the cosmetic holder 120. In one embodiment, the elastic or spring-loaded band can form the circumference of the cosmetic holder 120. For example, the cosmetic holder 120 can be a metal coil spring. The spring can stretch to fit around a cosmetic tool or product that is inserted into the aperture and can contract until the spring is in contact with the inserted cosmetic tool. The spring thus provides a secure fit around the inserted cosmetic tool.

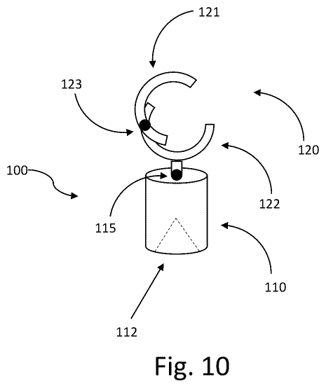

FIG. 10 is an illustration of a cosmetic holder 120 wherein the cosmetic holder 120 is formed by a first arm 121 and a second arm 122. The first arm 121 and the second arm 122 can be joined at a hinge point 123. In one embodiment, the hinge 123 can be a locking hinge (e.g., a self-locking hinge). In one example, the hinge can include a ratchet mechanism so that the first arm 121 and the second arm 122 can be fixed in place at incremental positions between a closed position and an open position. A closed position can refer to a position wherein the first arm 121 and the second arm 122 are in contact with or encircling an inserted cosmetic tool. An open position can refer to a position wherein at least one of the first arm 121 and the second arm 122 is not in contact with or encircling an inserted cosmetic tool. In one embodiment, the first arm 121 and the second arm 122 can meet or can overlap around an inserted cosmetic tool in the closed position in order to secure the cosmetic tool. In one embodiment, the first arm 121 and the second arm 122 can secure an inserted cosmetic tool in the closed position without overlapping with each other. For example, the first arm 121 and the second arm 122 can form an arc around a portion of the inserted cosmetic tool.

In one embodiment, the hinge 123 can be located along the length of the first arm rather than at the end of the first arm. Each end of the first arm 121 can extend past the hinge, as illustrated in FIG. 10. The first arm 121 and the second arm 122 can be in the same plane, or one arm can be stacked on top of the other arm. When the cosmetic holder 120 is open, a first end of the first arm 121 can extend into or towards the central opening (aperture) formed by the first arm 121 and the second arm 122. When a cosmetic tool is inserted into the opening, the cosmetic tool can push the first end of the first arm 121 to rotate the first arm 121 into a closed position. The first arm 121 can rotate freely until the inserted cosmetic tool is secured by both the first arm 121 and the second arm 122. In this manner, the user does not have to take extra steps to adjust the size of the aperture. The insertion of the cosmetic tool into the cosmetic holder 120 automatically rotates the arms of the cosmetic holder into a closed position to secure the cosmetic tool. In one embodiment, the cosmetic tool can be removed by moving the cosmetic tool along the central axis of the cosmetic holder 120 to slide the tool out of the cosmetic holder 120.

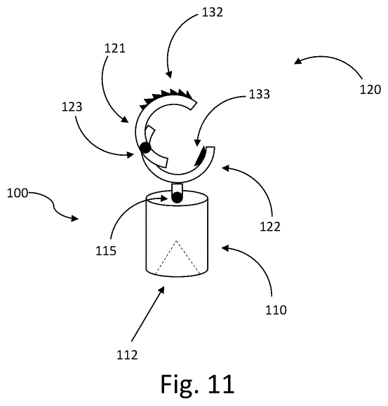

In one embodiment, the aperture of the cosmetic holder 120 can be adjusted via a ratcheting mechanism. FIG. 11 is an illustration of a first arm 121 of the cosmetic holder and a second arm 122 of the cosmetic holder forming a ratcheting mechanism. In one embodiment, the first arm 121 can include one or more teeth 130 along an outer surface or edge. In one embodiment, the one or more teeth can be asymmetrical. The second arm can include one or more structures configured to overlap with and engage the one or more teeth of the first arm. In one embodiment, the second arm can form a pawl 131 or click, wherein the edge of the pawl 131 can fit into a space between each of the one or more teeth of the first arm to fix the first arm to the second arm and lock the first arm and the second arm into place. The pawl can be spring-loaded. The aperture of the cosmetic holder 120 can be narrowed by pushing the first arm towards the second arm. In one embodiment, the second arm can include one or more divots, grooves, notches, or cutouts along the inner wall of the second arm, wherein the one or more teeth of the first arm can catch on the one or more structures in the second arm to fix the first arm to the second arm.

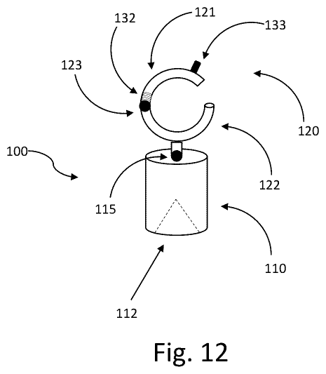

FIG. 12 is an illustration of a spring ring clasp formed by the first arm 121 and the second arm 122. The first arm 121 and/or the second arm 122 can be spring-loaded. In one embodiment, the second arm 122 can be hollow and the first arm 121 of the cosmetic holder can fit into the second arm 122 of the cosmetic holder. The first arm 121 can include an internal spring 132 at the hinge 123. The internal spring 132 is shown in FIG. 12 for illustrative purposes. The first arm 121 can also include a lever 133. When the lever 133 is pulled, the first arm can compress the spring 132 and open the cosmetic holder into a semicircle. In one embodiment, the first arm and the second arm can maintain the semicircle as a closed position when a cosmetic tool is inserted. The spring 132 can expand until the first arm secures, or is stopped by, the inserted cosmetic tool in the closed position. In this manner, the first arm can be held at a varying distance from the second arm to secure inserted cosmetic tools of varying sizes. In one embodiment, the lever 133 can be attached to a button or a handle to provide an easier actuation mechanism for a user with limited mobility.

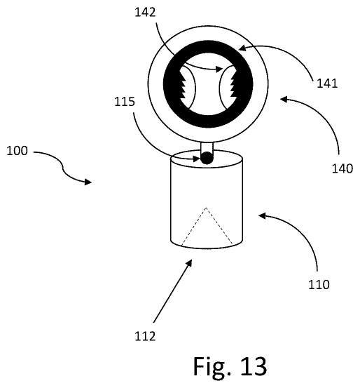

In one embodiment, the aperture of the cosmetic holder 120 can be adjusted by rotating the cosmetic holder. FIG. 13 is an illustration of a rotatable cosmetic holder 120. In one embodiment, the cosmetic holder 120 can include an outer shell 140 surrounding a ring 141, wherein the ring surrounds and is coupled to one or more inner jaws 142. The inner jaws 142 can be approximately parallel to the central axis of the outer shell and the ring or can be angled relative to the central axis. The inner surface of the ring 141 can form threads or teeth 143, wherein the threads or teeth can couple with corresponding threads or teeth on the sides of the one or more inner jaws 142. The corresponding threads on the sides of the one or more inner jaws can be angled relative to the central axis of the ring to form a tapered or conical screw-thread. When the outer shell 140 is rotated, the ring 141 also rotates. The threads 143 of the ring interlock with the tapered threads of the inner jaws so that the inner jaws 142 move laterally as a result of the rotational force applied to the ring. The lateral movement of the ring 141 along the angled threads pushes the inner jaws 142 to move closer together or further apart. For example, rotating the outer shell 140 clockwise can result in the inner jaws 142 being brought together, thus narrowing the aperture of the cosmetic holder 120. Rotating the outer shell 140 counterclockwise can result in the inner jaws 142 being pulled further apart, thus widening the aperture of the cosmetic holder 120. A cosmetic tool can be inserted in between the inner jaws, and the outer shell can be rotated in a first direction until the inner jaws are in contact with and securing the inserted cosmetic tool. The outer shell can then be rotated in a second, opposing direction to pull the inner jaws apart and remove the inserted cosmetic tool.

In one embodiment, the cosmetic holder 120 can include magnetic cores arrayed around a non-magnetic tube, which is filled with a magnetic fluid. Each of the magnetic cores can have arm portions that are surrounded by windings. The magnetic cores may be controlled by the control circuitry in the PCBA 1302 to act as a controllable active magnetic field-generating structure which is used to generate a variable magnetic field that acts upon the magnetic fluid, causing it to be displaced, as is described herein with reference to the electromagnetic positioner 1311 of FIG. 3B. The displacement of the fluid can result in the movement of a first arm or a second arm of the cosmetic holder 120 to a desired position to widen or narrow the aperture of the cosmetic holder 120.

In one embodiment, an inner surface of the cosmetic holder 120 can be lined with a flexible or elastic material, such as foam or rubber, as a padding. The inner lining of the cosmetic holder 120 can provide a flexible buffer to accommodate different sizes and shapes of cosmetic tools. The inner lining can secure an inserted cosmetic tool by fitting around and against the inserted cosmetic tool even if the cosmetic holder 120 is not in contact with the inserted cosmetic tool due to differences in size or shape. In one embodiment, the inner lining can be removably adhered or coupled to the inner surface of the cosmetic holder 120. In one embodiment, a number of inner linings of varying thicknesses can be provided for a cosmetic holder. In one embodiment, an inner lining can be exchanged for one that has an appropriate thickness matching an inserted cosmetic tool. The inner lining can be compatible with any of the cosmetic holder 120 embodiments presented in the disclosure.

In one embodiment, a portion of the cosmetic holder 120 can be connected to a button, switch, tab, strap, chain, lever, gear, or similar mechanical component. A user can adjust the aperture of the cosmetic holder 120 by engaging the button, switch, etc. or by pulling a strap or chain. In one example, a chain can be attached to or can form an arm of the cosmetic holder. Pulling the chain can rotate the arm to narrow the aperture of the cosmetic holder. Similarly, in some embodiments the cosmetic holder 120 can be connected to a mechanical component that can release or disengage the cosmetic holder. For example, a tab can be connected to a first arm of the cosmetic holder. Pushing down on the tab can cause a pushing force to be applied to the first arm to be rotated into an open position so that an inserted cosmetic tool can be removed. These mechanical components can be more easily engaged and actuated by users with limited mobility who may not be able to directly handle the cosmetic holder 120.

In one embodiment, the adjustment of the aperture can be automated via circuitry of the motion stabilization device 150 and/or the adapter 100. For example, the circuitry 703, 701 can use sensor data to determine a type of cosmetic tool that is being inserted into the cosmetic holder 120. The type of cosmetic tool can be associated with an aperture size. The cosmetic adapter 100 can include a motor, wherein the motor can be configured to rotate the first arm or the second arm of the cosmetic holder 120 in order to modify the aperture size. In one embodiment, the motor can be configured to rotate the ring of FIG. 13 to modify the aperture size. In one embodiment, the circuitry 703, 701 can receive a protocol associated with a type of cosmetic tool or an SKU associated with a certain cosmetic product based on the sensor data. The protocol can include an aperture size associated with the cosmetic tool or product. The cosmetic adapter 100 can then modify the aperture size based on the received protocol so that the cosmetic holder 120 fits around the cosmetic tool in a closed position. In one embodiment, the cosmetic adapter 100 can enlarge the cosmetic holder 120 into an open position when the inserted cosmetic tool is no longer being used. For example, pressure sensors in the cosmetic adapter 100 can indicate that an inserted cosmetic tool is not being used because the cosmetic tool is not in contact with the user. A motor can then enlarge the cosmetic holder 120, e.g., by rotating an arm of the cosmetic holder into an open position or by rotating the ring of FIG. 13 to move the inner jaws away from each other. The automatic opening of the cosmetic holder 120 can make it easier for the inserted cosmetic tool to be removed.

Obviously, numerous modifications and variations of the present invention are possible in light of the above teachings. It is therefore to be understood that within the scope of the appended claims, the invention may be practiced otherwise than as specifically described herein.

1. A cosmetic securement device for stabilization of a cosmetic applicator, comprising:
a motion stabilizer handle; and
an adapter coupled to a first end of the motion stabilizer handle;
wherein the motion stabilizer handle includes at least one sensor configured to detect a movement caused by a user,
circuitry configured to determine a compensation movement to offset the detected movement, and
at least one motion generation device configured to control a motion of the cosmetic applicator according to the determined compensation movement, and
wherein the adapter includes a cosmetic holder configured to hold a plurality of different types of cosmetic applicators, the cosmetic holder comprising a first arm and a second arm, the first arm and the second arm being connected at a hinge and configured to rotate around the hinge.

2. The device according to claim 1, wherein the first arm includes one or more teeth along an outer wall of the first arm.

3. The device according to claim 2, wherein the second arm includes one or more divots configured to couple with the one or more teeth of the first arm.

4. The device according to claim 2, wherein the second arm forms a pawl configured to engage the one or more teeth of the first arm.

5. The device according to claim 1, wherein the second arm is hollow and the first arm is configured to fit inside the second arm.

6. The device according to claim 1, wherein the first arm or the second arm is spring-loaded.

7. The device according to claim 1, wherein an inner wall of the cosmetic holder is lined with a padding material.

8. The device according to claim 1, further comprising a motor configured to rotate the first arm or the second arm around the hinge.

9. The device according to claim 1, wherein the first arm is rotated by movement of a magnetic fluid.

10. A cosmetic securement device for stabilization of a cosmetic applicator, comprising:
a motion stabilizer handle; and
an adapter including a cosmetic holder and coupled to a first end of the motion stabilizer handle;
wherein the motion stabilizer handle includes at least one sensor configured to detect a movement caused by a user,
circuitry configured to determine a compensation movement to offset the detected movement and an aperture of the cosmetic holder based on the cosmetic applicator, and
at least one motion generation device configured to control a motion of the cosmetic applicator according to the determined compensation movement, and
wherein the cosmetic holder is configured to hold a plurality of different types of cosmetic applicators.

11. The device according to claim 10, wherein the cosmetic holder includes a first arm and a second arm, the first arm and the second arm being connected at a hinge and configured to rotate around the hinge.

12. The device according to claim 11, further comprising a motor configured to rotate the first arm or the second arm around the hinge based on the determined aperture.

13. The device according to claim 11, wherein the first arm includes one or more teeth along an outer wall of the first arm.

14. The device according to claim 13, wherein the second arm includes one or more divots configured to couple with the one or more teeth of the first arm.

15. The device according to claim 13, wherein the second arm forms a pawl configured to engage the one or more teeth of the first arm.

16. The device according to claim 10, wherein the cosmetic holder includes a rotatable ring surrounding one or more internal jaws, wherein the rotatable ring is coupled to a tapered thread along the one or more internal jaws.

17. The device according to claim 10, further comprising a motor configured to rotate the rotatable ring based on the determined aperture.

18. A cosmetic securement device for stabilization of a cosmetic applicator, comprising:
a motion stabilizer handle; and
an adapter coupled to a first end of the motion stabilizer handle;
wherein the motion stabilizer handle includes at least one sensor configured to detect a movement caused by a user,
circuitry configured to determine a compensation movement to offset the detected movement, and
at least one motion generation device configured to control a motion of the cosmetic applicator according to the determined compensation movement, and
wherein the adapter includes a cosmetic holder configured to hold a plurality of different types of cosmetic applicators, the cosmetic holder comprising an adjustable ring.

19. The device according to claim 18, wherein the cosmetic holder includes a rotatable ring surrounding one or more internal jaws, wherein the rotatable ring is coupled to a tapered thread along the one or more internal jaws.

20. The device according to claim 18, wherein the adjustable ring is a circular spring.
